(19) United States
(12) Patent Application Publication
(10) Pub. No.: US 2010/0204792 A1
(43) Pub. Date: Aug. 12, 2010

(54) Permanently Adjustable Silicone Implant

(76) Inventor: Richard J. Greco, Savannah, GA (US)

Correspondence Address: NIXON & VANDERHYE, PC, 901 NORTH GLEBE ROAD, 11TH FLOOR, ARLINGTON, VA, 22203, US

(21) Appl. No.: 12/704,615

(22) Filed: Feb. 12, 2010

Related U.S. Application Data
(60) Provisional application No. 61/151,642, filed on Feb. 11, 2009.
(60) Provisional application No. 61/235,085, filed on Aug. 19, 2009.

(51) Int. Cl. A61F 2/12

(52) U.S. Cl. 623/8

(57) ABSTRACT

A surgically implantable breast prosthesis includes an outer shell defining a first interior volume filled with a liquid filler, and an inflatable inner bladder disposed in the first interior volume and defining a second interior volume. The inflatable inner bladder is selectively fillable with the liquid filler via a valve assembly accessible from an exterior of the outer shell. The valve assembly is substantially non-palpable and includes identifying structure that is discoverable using an external device. The valve assembly includes an injection port that provides access to the inflatable inner bladder such that a size of the inflatable inner bladder is adjustable.

### CROSS-REFERENCES TO RELATED APPLICATIONS

This application claims the benefit of U.S. Provisional Patent Application Ser. No. 61/151,642, filed Feb. 11, 2009 and U.S. Provisional Patent Application Ser. No. 61/235,085, filed Aug. 19, 2009, the entire contents of each of which is herein incorporated by reference.

### STATEMENT REGARDING FEDERALLY SPONSORED RESEARCH OR DEVELOPMENT

(NOT APPLICABLE)

### BACKGROUND OF THE INVENTION

Silicone implants have many advantages over saline implants. Presently available adjustable implants have short term advantages because they allow for intra operative and early postoperative adjustments. All present adjustable implants are saline implants (although manufacturers have adjustable silicone implants in progress), and they all require the removal of a port at some time one day to one year later.

These devices have the disadvantages of an external port that is palpable, that they require a second operation to remove or cap the adjustable system, and that they have the risk of deflation from the capped line or valve from the removed system. In addition, there is an increased risk of infection associated with a second operation. Previous studies actually demonstrated higher incidences of both deflation and infection associated with previous attempts at permanent adjustable implants.

Tissue expanders have been valuable devices for the reconstruction of breasts, and they work extremely well with integral ports—although they are palpable and have all the characteristics of saline implants—which have too many ripples for breast reconstruction.

One of the largest problems with implant placement has been the need for reoperation. In a recent study by Allergan, the reoperation rate was close to 25% over 5 years. The most common reasons are size change, asymmetry, capsular contracture, hematoma, and infection. Implant design can not solve hematoma, infection, or exact placement of implants. This implant design concept may be able to deal with four separate problems that would otherwise require a second operative procedure to correct.

1) Dissatisfaction—Rarely do women choose to change their implants to have smaller implants, but recent studies by Mentor and by Casas show that 25-33% of women choose to have a reoperation to increase the size of their implant—usually by about 100 cc.

2) Breast asymmetry—when different sized silicone implants are used to make up for a pre operative breast size asymmetry, they become less symmetrical as the larger breast grows more than the smaller breast if the patient gains weight

3) Breast Reconstruction—changes in the native breast with time—usually leads to a larger native breast than the implant reconstructed breast. Some equalization via an adjustable silicone implant would reduce the need for a second operation on the reconstructed breast.

4) Capsular Contracture—2-8% of individuals suffer from capsular contracture. This is sometimes treated by closed capsulotomy, and usually by operative intervention. A paper in Europe demonstrated that overexpansion for two months and then release of the volume lead to less capsular contractures. In patients that develop a contracture, it is proposed to treat this by stretching the capsule by a temporary internal expansion of the implant and stretching—as pregnancy does to the abdomen.

### SUMMARY OF THE INVENTION

It would be desirable to have a silicone device that was not only adjustable, but jumps over the present concept of an adjustable silicone implant that requires an operation to remove the palpable port, and could be adjusted years later when needed.

In an exemplary embodiment, a surgically implantable breast prosthesis includes an outer shell defining a first interior volume, and an inflatable inner bladder disposed in the first interior volume and defining a second interior volume. A liquid filler is disposed in the first interior volume and the second interior volume. A valve assembly accessible from an exterior of the outer shell and sealing fluid communication to the second interior volume is substantially non-palpable and is identifiable using an external device. The valve assembly provides access to the inflatable inner bladder such that a size of the inflatable inner bladder is adjustable.

The external device may be one or more of a fluoroscopic device, an ultrasonic device, a magnetic device, an electromagnetic device, or the like. Preferably, the valve assembly is sized and positioned to enable permanent adjustability of the inflatable inner bladder. In one arrangement, the inflatable inner bladder is disposed centrally within the first interior volume. In an alternative arrangement, the outer shell is a shaped device including a superior pole and an inferior pole, wherein the inflatable inner bladder is disposed in the inferior pole.

The inflatable inner bladder preferably has a volume of at least 100 cc and/or up to 35% of a volume of the outer shell.

The valve assembly may include an injection port that is substantially flush with an exterior surface of the outer shell. The injection port may comprise a self-sealing surface. In addition, the injection port may comprise a metal back that serves as stop for an injection needle.

In one arrangement, the breast prosthesis includes a second inflatable inner bladder disposed in the first interior volume and defining a third interior volume. In this context, the valve assembly includes an injection port in fluid communication with the third interior volume. The outer shell may be shaped to include an upper pole and a lower pole, wherein one of the inflatable inner bladders is disposed in the upper pole and the other of the inflatable inner bladders is disposed in the lower pole. The injection port may be provided with a partition including an upper port and a lower port, wherein the upper port is in fluid communication with one of the inflatable inner bladders and the lower port is in fluid communication with the other of the inflatable inner bladders.

In another exemplary embodiment, a surgically implantable breast prosthesis includes an outer shell defining a first interior volume filled with a liquid filler, and an inflatable inner bladder disposed in the first interior volume and defining a second interior volume. The inflatable inner bladder is selectively fillable with the liquid filler via a valve assembly accessible from an exterior of the outer shell. The valve assembly is substantially non-palpable and includes identifying structure that is discoverable using an external device. The valve assembly includes an injection port that provides access to the inflatable inner bladder such that a size of the inflatable inner bladder is adjustable.

In still another exemplary embodiment, a method of adjusting a size of the implantable breast prosthesis of the described embodiments includes the steps of identifying the valve assembly with the external device, and injecting or extracting the liquid filler from the inflatable inner bladder via the valve assembly until the inflatable bladder is a desired size.

### BRIEF DESCRIPTION OF THE DRAWINGS

These and other aspects and advantages will be described in detail with reference to the accompanying drawings, in which:

FIG. 1 is a perspective view of an exemplary implant according to the described embodiments; and

FIG. 2 is a perspective view of an exemplary implant according to an alternative embodiment.

### DETAILED DESCRIPTION OF THE INVENTION

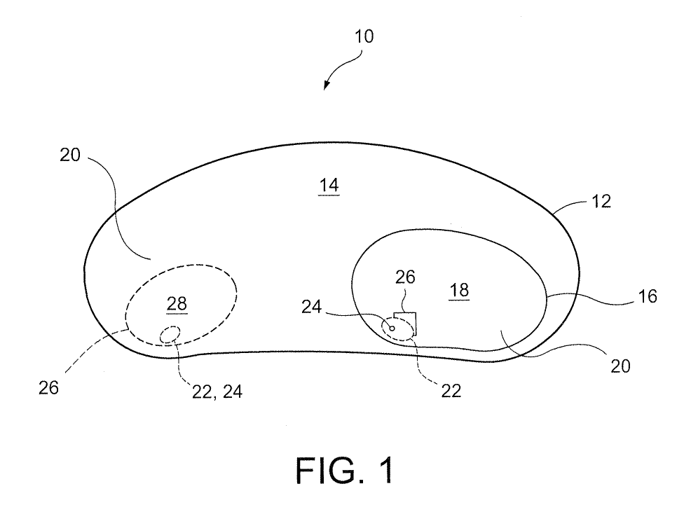

With reference to FIG. 1, a surgically implantable breast prosthesis 10 includes an outer shell 12 defining a first interior volume 14, and an inflatable inner bladder 16 disposed in the first interior volume and defining a second interior volume 18. A liquid filler 20 is disposed in the first interior volume and the second interior volume. A valve assembly 22 accessible from an exterior of the outer shell 12 and sealing fluid communication to the second interior volume 18 is substantially non-palpable and is identifiable using an external device. The valve assembly 22 provides access to the inflatable inner bladder 16 such that a size of the inflatable inner bladder is adjustable.

The external device may be any suitable device, including without limitation, one or more of a fluoroscopic device, an ultrasonic device, a magnetic device, an electromagnetic device, or the like. Preferably, the valve assembly 22 is sized and positioned to enable permanent adjustability of the inflatable inner bladder 16. In one arrangement, the inflatable inner bladder 16 is disposed centrally within the first interior volume 14. In an alternative arrangement, the outer shell 12 is a shaped device including a superior pole and an inferior pole, wherein the inflatable inner bladder 16 is disposed in the inferior pole.

The valve assembly 22 preferably includes an injection port 24 that is substantially flush with an exterior surface of the outer shell 12. The injection port 24 may comprise a self-sealing surface. In addition, the injection port 24 may comprise a metal back 26 that serves as stop for an injection needle.

The breast prosthesis 10 may include a second inflatable inner bladder 26 disposed in the first interior volume 14 and defining a third interior volume 28. In this context, the valve assembly includes 22 an injection port 24 in fluid communication with the third interior volume 28. If the outer shell 12 is shaped to include an upper pole and a lower pole, one of the inflatable inner bladders 16, 26 can be disposed in the upper pole and the other of the inflatable inner bladders 16, 26 can be disposed in the lower pole. The injection port 24 may be provided with a partition including an upper port and a lower port, wherein the upper port is in fluid communication with one of the inflatable inner bladders and the lower port is in fluid communication with the other of the inflatable inner bladders.

A preferred embodiment of this implant is to have a silicone implant, with either a smooth or textured surface, that has an inflatable bladder in the device, which is approached through an integral port small enough to be either non-palpable, or barely palpable (i.e., “substantially non-palpable”). The expandable bladder could be of any size, but probably should be a minimum of 100 cc and up to a volume equal to 35% of the present implant volume. The silicone implant could be made of any of the types of silicone polymers available. Alternatively, in a saline version, a saline base implant may include a self sealing valve leading to the original volume and base implant. The implant would have a non-palpable valve attached to an interior bladder that can be adjusted transcutaneously.

In tissue expanders, large diameter and purposely palpable ports are used to make it easier for the surgeon to identify the port and to expand the device many times. It is understood that the tissue expander will be removed. There are many new technologies for allowing identification of the integral port—allowing the design of a much smaller port that needs to be entered infrequently. With regard to valve designs, there are multiple valve designs presently used. Most are designed to be temporary and nonpalpable—such as the saline implant valve which is meant to be removed, and to be self sealing. Others are meant to be large and palpable so that it can remain—but the palpability is unacceptable for cosmetic purposes.

The port could have a separate surface from the silicone shell. It will be flush with the device so as not to create a visible bulge. As noted, the device can be identified by one of many modalities including fluoroscopic assistance, ultrasonic assistance, or a new novel new approach where one can transfer energy by transcutaneous excitement of a ring around the port, and using a device that identifies this energy/magnetism for needle placement.

During the life of any woman who has required implant reconstruction for asymmetry or breast cancer, the need to adjust is rare, but may become a reality. The ability to perform a long term adjustment would be extremely valuable—often avoiding the need for an additional operation for an implant exchange for size or asymmetry. In addition, temporary expansion to rupture or stretch a capsular contracture may avoid the need for a second operation.

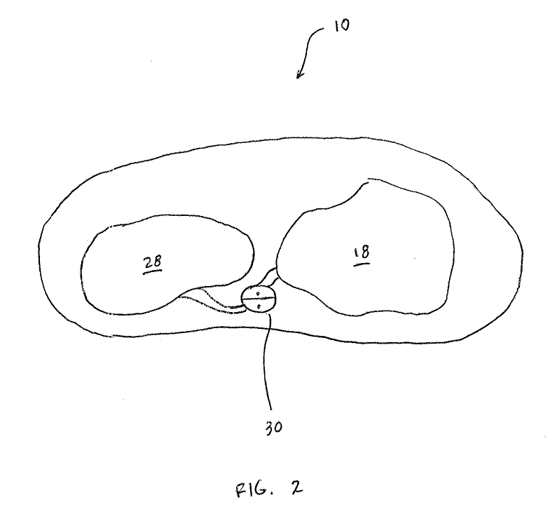

Moreover, the implant could have multiple adjustable components and is not limited to one or any specific number. The valve devices could have a partition/split 30 (see FIG. 2) so that one valve could be entered in the upper quadrant for the upper bladder and the lower quadrant for the lower bladder. The device could have multiple valves or one valve with multiple partitions.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A surgically implantable breast prosthesis comprising:
an outer shell defining a first interior volume;
an inflatable inner bladder disposed in the first interior volume and defining a second interior volume;
a liquid filler disposed in the first interior volume and the second interior volume; and
a valve assembly accessible from an exterior of the outer shell and sealing fluid communication to the second interior volume, wherein the valve assembly is substantially non-palpable and is identifiable using an external device, the valve assembly providing access to the inflatable inner bladder such that a size of the inflatable inner bladder is adjustable.

2. A surgically implantable breast prosthesis according to claim 1, wherein the external device comprises at least one of a fluoroscopic device and an ultrasonic device.

3. A surgically implantable breast prosthesis according to claim 1, wherein the external device comprises at least one of a magnetic device and an electromagnetic device.

4. A surgically implantable breast prosthesis according to claim 1, wherein the valve assembly is sized and positioned to enable permanent adjustability of the inflatable inner bladder.

5. A surgically implantable breast prosthesis according to claim 1, wherein the inflatable inner bladder is disposed centrally within the first interior volume.

6. A surgically implantable breast prosthesis according to claim 1, wherein the outer shell is a shaped device including a superior pole and an inferior pole, and wherein the inflatable inner bladder is disposed in the inferior pole.

7. A surgically implantable breast prosthesis according to claim 1, wherein the inflatable inner bladder has a volume of at least 100 cc.

8. A surgically implantable breast prosthesis according to claim 7, wherein the inflatable inner bladder has a maximum volume equal to 35% of a volume of the outer shell.

9. A surgically implantable breast prosthesis according to claim 1, wherein the valve assembly comprises an injection port, and wherein the injection port is substantially flush with an exterior surface of the outer shell.

10. A surgically implantable breast prosthesis according to claim 9, wherein the injection port comprises a self-sealing surface.

11. A surgically implantable breast prosthesis according to claim 10, wherein the injection port comprises a metal back that serves as stop for an injection needle.

12. A surgically implantable breast prosthesis according to claim 1, comprising a second inflatable inner bladder disposed in the first interior volume and defining a third interior volume, wherein the valve assembly comprises an injection port in fluid communication with the third interior volume.

13. A surgically implantable breast prosthesis according to claim 12, wherein the outer shell is shaped to include an upper pole and a lower pole, and wherein one of the inflatable inner bladders is disposed in the upper pole and the other of the inflatable inner bladders is disposed in the lower pole.

14. A surgically implantable breast prosthesis according to claim 12, wherein the injection port comprises a partition including an upper port and a lower port, and wherein the upper port is in fluid communication with one of the inflatable inner bladders and the lower port is in fluid communication with the other of the inflatable inner bladders.

15. A surgically implantable breast prosthesis comprising:
an outer shell defining a first interior volume filled with a liquid filler; and
an inflatable inner bladder disposed in the first interior volume and defining a second interior volume, the inflatable inner bladder being selectively fillable with the liquid filler via a valve assembly accessible from an exterior of the outer shell, wherein the valve assembly is substantially non-palpable and includes identifying structure that is discoverable using an external device, the valve assembly including an injection port that provides access to the inflatable inner bladder such that a size of the inflatable inner bladder is adjustable.

16. A surgically implantable breast prosthesis according to claim 15, wherein the valve assembly is sized and positioned to enable permanent adjustability of the inflatable inner bladder.

17. A surgically implantable breast prosthesis according to claim 15, comprising a second inflatable inner bladder disposed in the first interior volume and defining a third interior volume, wherein the injection port is in fluid communication with the third interior volume.

18. A surgically implantable breast prosthesis according to claim 17, wherein the outer shell is shaped to include an upper pole and a lower pole, and wherein one of the inflatable inner bladders is disposed in the upper pole and the other of the inflatable inner bladders is disposed in the lower pole.

19. A method of adjusting a size of the implantable breast prosthesis of claim 1, the method comprising identifying the valve assembly with the external device, and injecting or extracting the liquid filler from the inflatable inner bladder via the valve assembly until the inflatable bladder is a desired size.
